(19) United States
(12) Patent Application Publication
(10) Pub. No.: US 2021/0046003 A1
(43) Pub. Date: Feb. 18, 2021

(54) HEALTH DRINK COMPOSITION FOR PREVENTING LIVER DAMAGE CAUSED BY ALCOHOL WHILE PROVIDING EXCELLENT HANGOVER RELIEF

(71) Applicant: GINSENG BYPHARM CO., LTD., Gangwon-do (KR)

(72) Inventor: Jun-Min AN, Gangwon-do (KR)

(21) Appl. No.: 16/979,681

(22) PCT Filed: Apr. 12, 2019
(86) PCT No.: PCT/KR2019/004462

§ 371 Date: Sep. 10, 2020

(30) Foreign Application Priority Data
Sep. 17, 2018 (KR) 10-2018-0110814

(51) Int. Cl. A61K 9/00; A23L 2/52; A23L 2/38; A61K 36/45; A61K 36/725; A61K 36/87; A61K 36/06; A61P 1/16; A23L 33/105; A23L 33/14; A61K 36/258

(52) U.S. Cl. CPC A61K 9/0095; A23L 2/52; A23L 2/38; A61K 36/45; A61K 36/725; A61K 36/258; A61K 36/06; A61P 1/16; A23L 33/105; A23L 33/14; A61K 36/87

(57) ABSTRACT

A drink composition for preventing alcohol-induced liver damage, which is effective for hangover relief and which contains 2 to 4 wt % of black ginseng, 4 to 7 wt % of blueberry, 0.3 to 1 wt % of dry yeast, 0.5 to 1.5 wt % of jujube pulp, and 2 to 4 wt % of oriental raisin tree fruit and is produced by hot-water extraction using the remaining wt % of purified water. The composition has a liver damage suppressing effect, reduces hepatitis activity index, and thus may be used as a composition capable of preventing or treating various liver diseases. Furthermore, it has very excellent effects on alcohol degradation and acetaldehyde degradation, alleviates headache and vomiting symptoms the next day after drinking, and thus is also very useful as a composition for liver protection or hangover relief, which is taken before and after alcohol drinking.

### BACKGROUND

The present invention relates to a composition for preventing or treating alcohol-induced liver damage, which is effective for hangover relief, and more specifically to a composition for preventing or treating alcohol-induced liver damage, which is effective for hangover life and which contains black ginseng, blueberry, dry yeast, jujube pulp, and oriental raisin tree fruit.

The liver is a central organ that metabolizes carbohydrates, protein and fat, and is an important organ responsible for the function of excreting substances in the body through oxidation, reduction, hydrolysis and conjugation reactions. However, since liver diseases are found after they are considerably exacerbated without causing pain or subjective symptoms in early stages, the liver is known as a ‘silent organ.’

As liver diseases are exacerbated, constipation, irritable bowel syndrome, or a change in stool color may appear, and patients with liver damage may have persistent vomiting and undergo sudden weight loss.

In addition, due to impaired liver function, protein production in the body may be hampered and body fluids with impaired circulation ability may be lowered by gravity to cause circulatory problems, such as swelling of the legs, ankles and feet. In addition, the most common symptoms of liver damage may occur, such as fatigue, chronic fatigue, and decreased memory.

Signs of severe liver damage include abdominal swelling caused by ascites accumulation in the abdominal cavity due to high albumin and protein levels in the blood and body, and jaundice, which causes yellowing of the skin and eyes, is also a sign of liver damage.

In addition, there may be pain in the upper right abdomen or in the lower right part of the chest (pain in the liver), and urine may turn dark yellow due to increased bilirubin levels in the bloodstream, and skin itching or peeling may also occur.

The major causes of liver damage include hepatitis virus (A, B, C, etc.), alcohol drinking, and non-alcoholic fatty liver. According to statistics, the treatment cost for alcoholic liver disease in Korea has been increasing rapidly every year. In addition, when analyzing the statistics provided by a domestic life insurance company, the number of deaths from alcoholic liver disease among the causes of death rapidly increased 7 times compared to 10 years ago.

Alcoholic liver disease caused by continuous alcohol consumption is emerging as a social problem worldwide, including in Korea. Especially, if alcoholic fatty liver and hepatitis persist, they can lead to the stage of liver fibrosis and liver cirrhosis which cannot be reversed, and thus liver transplantation remains the only treatment option.

Meanwhile, according to the National Cancer Center statistics, 60% to 70% of hepatitis virus carriers in Korea are hepatitis B carriers, and in particular, 75% of patients with hepatitis B are hepatitis B carriers, and 5% to 10% of the total population has chronic hepatitis B virus infection. In addition, hepatitis B and C virus infection and various chronic diseases of the liver cause liver cancer which is the most frequent malignant tumor in the world.

Conventional agents that are currently used for the treatment of liver diseases include liver function supplements, antiviral agents, hepatocyte promoters, immunosuppressants, fibrosis inhibitors, biphenyl dimethyl dicarboxylate, interferons, and the like. However, it is known that these agents have low therapeutic effects and adverse effects and co-administration of these agents to supplement them not only increases the therapeutic effect, but also shows adverse effects. Therefore, there is a need for research and development of functional raw materials for improving liver function, which have little adverse effects.

The drinking rate of Korean adult males over 20 years old is very high at 83.3%, and the drinking rate of Korean adult females is also 54.9% and rapidly increasing, and Korean adults are seriously exposed to excessive drinking due to wrong drinking culture. The damage caused by excessive alcohol consumption not only causes various diseases including liver disease, but also adversely affects the emotions and lives of individuals, families, and society, causing huge economic and social losses nationwide.

Excessive drinking may cause various adverse effects, such as thirst, general malaise, fatigue, memory loss, abdominal distension, indigestion, vomiting, diarrhea, and vitamin deficiency, and also increases the risk of alcoholism.

Among liver diseases, diseases caused by chronic drinking are largely divided into alcoholic fatty liver, alcoholic hepatitis, and alcoholic cirrhosis. However, it is rare for a person to have only one of these diseases, and each disease is manifested to varying degrees in each individual.

Chronic drinking is the most common cause of cirrhosis in the United States and is the second most common cause of cirrhosis after viral hepatitis in Korea.

In normal alcohol metabolism, when alcohol is consumed, it is absorbed in the stomach or small intestine, 10% of the absorbed alcohol is excreted in breath, sweat, urine, and the like, and the remaining 90% of alcohol enters the blood vessels and is metabolized in the liver (M Nakanishi; Saishin Igaku, 31, p2086, 1976). Subsequently, alcohol dehydrogenase (ADH) present in the liver cells oxidizes the alcohol to acetaldehyde, and the acetaldehyde is degraded to acetic acid by acetaldehyde dehydrogenase in the liver cells, and the acetic acid is then transferred to the muscles or adipose tissue of the whole body and is finally decomposed into carbon dioxide gas and water.

However, if this metabolic process is not smooth due to excessive drinking, acetaldehyde accumulates, resulting in cerebral vasoconstriction leading to heavy head, as well as hangovers such as headache or heartburn.

Individually recognized functional raw materials useful for liver health, which help solve these problems, include milk thistle extract, broccoli sprout powder, shiitake mushroom mycelium extract, raspberry extract powder, bellflower extract, and the like, and functional ingredients that help protect the liver from alcoholic damage include oriental raisin tree extract and lactic acid bacteria fermented kelp extract (Ministry of Food and Drug Safety, 2018).

Conventional arts related to compositions for liver protection include Korean Patent No. 10-0477957 (entitled “Novel pyrrole derivative having liver protective activity isolated from Chinese matrimony vine and a composition containing the same”), Korean Patent No. 10-0633851 (“Composition for preventing liver or preventing or treating liver disease containing a heat and dried Allium victorialis extract as an active ingredient”), Korean Patent No. 10-1106499 (Food composition effective for liver protection containing an extract of young branches of oriental raisin tree”), Effect of Protaetia brevitarsis larva on recovery on liver toxicity (Jeong-Soo Jeon et al., Journal of Sericultural and Entomological Science; 50(2), p93, 2012), Effects of Allomyrina dichotoma on liver protection and liver cell regeneration (Eun-Ji Lee et al., Korean Journal of Life Science; 25(3), p307, 2015), Effect of cricket on liver protection (Mi-Young Ann, Korean Journal of Food Science and Technology; 34(4), p684, 2002), and the like.

Regarding hangover relief, patent documents, including Korean Patent Application Publication No. 2002-0081995 (entitled “Agent for hangover relief and liver function improvement by blended food and a preparation method therefor”), and Korean Patent Application Publication No. 2002-0064151 (entitled “Lower alcohol insoluble extraction fraction and polysaccharide substance having liver toxicity- and hangover-relieving activity isolated from oriental raisin tree and a composition containing the same”), report that various plant-derived agents, including rice embryo bud extract, alder tree extract, oak wood vinegar, Pueraria flos extract, and milk thistle extract, are effective against hangovers. However, these agents are effective only in some of the various changes caused by drinking, or have insignificant effects.

### SUMMARY OF THE INVENTION

Therefore, a problem to be solved by the present invention is to provide a drink composition for preventing alcohol-induced liver damage, which is effective for hangover relief.

In order to accomplish the above technical problem, the present invention provides a drink composition for preventing alcoholic liver disease or relieving hangover, which contains 2 to 4 wt % of black ginseng, 4 to 7 wt % of blueberry, 0.3 to 1 wt % of dry yeast, 0.5 to 1.5 wt % of jujube pulp, and 2 to 4 wt % of oriental raisin tree fruit and is produced by hot-water extraction using the remaining wt % of purified water.

The drink composition for preventing alcoholic liver disease or relieving hangover may further contain 2 to 4 wt % of green onion root and be produced by hot-water extraction.

The drink composition for preventing alcoholic liver disease or relieving hangover may further contain 2 to 4 wt % of radish greens and be produced by hot-water extraction.

In the drink composition for preventing alcoholic liver disease or relieving hangover, the alcoholic liver disease may be alcoholic hepatitis, alcoholic fatty liver, or alcoholic liver cirrhosis.

The composition of the present invention as described above has a liver damage suppressing effect, reduces hepatitis activity index, and thus may be used as a composition capable of preventing or treating various liver diseases. In addition, it has very excellent effects on alcohol degradation and acetaldehyde degradation, alleviates headache and vomiting symptoms the next day after drinking, and thus is also very useful as a composition for liver protection or hangover relief, which is taken before and after alcohol drinking.

### DETAILED DESCRIPTION OF THE INVENTION

The present invention will be described in detail below.

The present invention provides a health drink composition for preventing alcohol-induced liver damage, which is effective for hangover relief and which contains 2 to 4 wt % of black ginseng, 4 to 7 wt % of blueberry, 0.3 to 1 wt % of dry yeast, 0.5 to 1.5 wt % of jujube pulp, and 2 to 4 wt % of oriental raisin tree fruit and is produced by hot-water extraction using the remaining wt % of purified water.

The black ginseng, obtained by nine cycles of steaming and drying of ginseng, has an increased content of ginsenoside Rg3, which is the main component of red ginseng, and thus has better cholesterol controlling ability than ginseng and red ginseng. The black ginseng has been proved and reported to have a very excellent preventive effect against adult diseases classified as incurable in modern medicine. In addition, the black ginseng has a significantly higher absorption rate of saponin than red ginseng, and has various effects such as memory improvement and fatigue recovery. In the present invention, any black ginseng may be used without particular limitation, as long as it is widely known in the art to which the present invention pertains. The black ginseng in the present specification also includes fermented black ginseng.

The blueberry is a kind of bilberry, and 150 to 200 blueberry species are distributed worldwide in the Northern Hemisphere. There are three main varieties: lowbush blueberry, highbush blueberry, and rabbiteye blueberry. The blueberry contains anthocyanin pigments symbolized by blue color, sweet and sour sugars, and viscous pectin, and has a subtle scent. The blueberry contains 4.5 g of dietary fiber per 100 g, and is rich in calcium, iron, manganese, and the like. As blueberry-related products, candy, chewing gum, jam, drinks and the like are being produced worldwide. In recent years, the development of functional foods and medicines for improving the functionality of eyes has been actively made.

The dry yeast includes active dry yeast and inactive dry yeast.

The active dry yeast is used for the production of, for example, bread or crackers, and is obtained by drying yeast to a moisture content of 8% or less with dry air at about 30 to 40° C. so that the yeast does not die. Since the active dry yeast can maintain its performance for several months at room temperature, it is used in bakeries located at a distance, or for household gods or export products. It is generally obtained by rotary drying or band drying and finished in a granular form or a short-rod form. In some cases, it is obtained in a powder form by spray drying. The inert dry yeast includes yeasts for medicinal, food and feed purposes. The yeast for medicinal purposes is mainly aimed at the use of vitamins, amino acids, minerals, etc., contained in yeast cells, but the yeasts for food and feed purposes are mainly aimed at the use of cell proteins. In the present invention, the active dry yeast and the inert dry yeast may be without distinction, because the drink composition is produced as a hot-water extract.

The jujube pulp is obtained by removing the seed from jujube, it has a sweet taste when ripens red, and may be eaten raw. In addition, it may be dried thoroughly after harvesting and used as dry fruit for confectionery, cooking and medicinal purposes. Jujube is processed in daily life and used as jujube wine, jujube tea, jujube vinegar, jujube porridge, etc. Honey jujube as a processed product is popular even in China, Japan and Europe. In oriental medicine, jujube is used as a diuretic, a tonic or a mitigative.

The oriental raisin tree fruit refers to the stalked fruit of Hovenia dulcis Thunb. belonging to the family Rhamnaceae. It has a little odor, is bitter and astringent in taste, is used against fever, dry mouth, hiccups, vomiting, etc., and is known to help diuresis and treat a liver damaged by alcoholism.

According to the present invention, the components described above are added to purified water, subjected to hot-water extraction by heating, and filtered, thereby making a drink. Technology for all the processes for making the drink may be used without particular limitation, as long as it is widely known in the art to which the present invention pertains.

The present invention may also provide a health drink composition for preventing alcohol-induced liver damage, which is effective for hangover relief, in which the health drink composition further contains 2 to 4 wt % of green onion root and is produced by hot-water extraction.

The green onion has high contents of calcium, salt, vitamins, and the like, and has a peculiar scent, and thus it may be eaten raw and is widely used for medicinal and cooking purposes. In addition, the green onion is a plant whose leaf, stem and root may all be eaten, and in oriental medicine, the root and stem of the green onion are used for the treatment of diseases such as fever, headache, diarrhea, and eye diseases, based on the antibacterial properties thereof. In the present invention, the green onion root is used to provide a synergistic effect on alcohol degradation and metabolism.

The present invention may also provide a health drink composition for preventing alcohol-induced liver damage, which is effective for hangover relief, in which the health drink composition further contains 2 to 4 wt % of radish greens and is produced by hot-water extraction.

The term “radish greens” refers to the leaf and stem of radish. It is known that the radish greens are rich in vitamins A, B1, B2, C and K, calcium, iron, amino acids, and the like, contain, inter alia, carotene and chlorophyll, from which vitamin A originates, and thus act to promote hematopoiesis and perform important physiological actions, including hemorrhoid treatment and anti-allergic activities for cell revival. In the present invention, the radish greens are used to provide a synergistic effect on alcohol degradation and metabolism. The present invention provides a composition for liver protection or hangover relief, which contains a hydrogen-absorbing metal as an active ingredient and optionally further contains vitamin C. In addition, the composition according to the present invention is provided as a pharmaceutical composition or a health functional food for preventing or treating an alcoholic liver disease represented by alcoholic hepatitis, alcoholic fatty liver or alcoholic liver cirrhosis.

The present invention will be described in detail below with reference to Examples.

1. Production of Drink Compositions

Drinks having the compositions shown in Table 1 were produced and used in the experiment described below. It was confirmed that the hangover-relieving ability of black ginseng significantly increased compared to those of other components. Thus, a composition containing an extract of black ginseng was set as a Comparative Example.

After extraction with stirring at 95 to 100° C. for 3 hours, the extracts were filtered, thereby producing drink compositions.

TABLE 1

| | | | | | | | Comparative | |
|---|---|---|---|---|---|---|---|---|
| | Example 1 | | Example 2 | | Example 3 | | Example | |
| Black ginseng | 3 | wt % | 3 | wt % | 3 | wt % | 15 | wt % |
| Blueberry | 6 | wt % | 6 | wt % | 6 | wt % | | |
| Dry yeast | 0.5 | wt % | 0.5 | wt % | 0.5 | wt % | | |
| Jujube pulp | 1 | wt % | 1 | wt % | 1 | wt % | | |
| Oriental raisin | 3 | wt % | 3 | wt % | 3 | wt % | | |
| tree fruit | | | | | | | | |
| Green onion | | | 2 | wt % | | | | |
| root | | | | | | | | |
| Radish greens | | | | | 2 | wt % | | |
| Purified water | 86.5 | wt % | 84.5 | wt % | 84.5 | wt % | 85 | wt % |

<Experimental Example: Establishment of Alcohol-Administered Animal Models and Administration of Materials>

As experimental animals, Sprague-Dawley male white rats (weighed 160 to 180 g) were purchased from Daehan Bio Link Co., Ltd. (Eum-sung gun, Republic of Korea). The rats were acclimated to laboratory conditions at a temperature of 20±2° C. and a relative humidity of 55±1% (RH) for 1 hour under 12-hr light/12-hr dark cycles.

For used as a control, experimental animal solid feed (PMI Nutrition International) was ground to a particle size of 70 mesh by a roll mill, and then granulated into granules having a hardness of 30 kg/cm by a rotary granulator, followed by drying. For use in experimental groups, experimental animal solid feed (PMI Nutrition International) was mixed with the composition of each Example at a weight ratio of 5:5, and then granulated in the same manner as the control.

The general feed granules and the granules containing the composition of each Example, prepared as described above, were freely fed to experimental animals (n=8 per group).

For ethanol administration, a 25% (v/v) aqueous solution of ethanol was orally administered daily through the stomach tube at a level of 5 mL/kg body weight/day for 6 weeks. That is, ethanol was not administered during one week of the 8-week breeding period to provide acclimation to the new mixed powder feed, and was then administered for 6 weeks, and was not administered for the last 1 week.

The experimental treatments are as shown in Table 2 below, and the results of evaluating the state of the subsequent experimental animals are indicated according to the classification of Table 2 below.

TABLE 2

| Classification | Groups |
|---|---|
| Control group | Administered with general feed granules |
| Group administered with | Administered with 25% (v/v) ethanol aqueous |
| Comparative Example | solution + general feed granules |
| Group administered | Administered with 25% (v/v) ethanol aqueous |
| with Example 1 | solution + mixed granules of general feed and |
| | Example 1 |
| Group administered | Administered with 25% (v/v) ethanol aqueous |
| with Example 2 | solution + mixed granules of general feed and |
| | Example 2 |
| Group administered | Administered with 25% (v/v) ethanol aqueous |
| with Example 3 | solution + mixed granules of general feed and |
| | Example 3 |
| Group administered | Administered with 25% (v/v) ethanol aqueous |
| with Comparative | solution + mixed granules of general feed and |
| Example | Comparative Example |

<Analysis of Body Weight Change>

For all the animals used in the experimental example, symptoms were observed once a day. The body weight was measured using an animal weight scale every 5 days at a given time, and the results are shown in Table 3 below.

TABLE 3

| Classification | Start | 5 | 10 | 15 | 20 | 25 | 30 |
|---|---|---|---|---|---|---|---|
| Control group | 169 g | 175 g | 212 g | 227 g | 232 g | 240 g | 255 g |
| Group administered | 168 g | 170 g | 181 g | 192 g | 209 g | 217 g | 221 g |
| with Comparative | | | | | | | |
| Example | | | | | | | |
| Group administered | 169 g | 173 g | 207 g | 222 g | 228 g | 234 g | 250 g |
| with Example 1 | | | | | | | |
| Group administered | 170 g | 174 g | 211 g | 225 g | 229 g | 237 g | 252 g |
| with Example 2 | | | | | | | |
| Group administered | 170 g | 174 g | 210 g | 225 g | 230 g | 238 g | 253 g |
| with Example 3 | | | | | | | |

No animals died during this experimental period, and no animals with specific clinical symptoms were observed. Referring to Table 3 above, the body weight increased in all groups during the experimental period, but the group administered with the Example showed an increase similar to the control group. However, in the group administered with the Comparative Group, the degree of weight gain was very slow.

<Results of Blood Biochemical Analysis>

After completion of the experiment, the blood collected from the abdominal artery was allowed to stand at room temperature for 30 minutes, and then centrifuged at 4° C. for 15 minutes at 3,000 rpm to separate the serum. Then, enzymes in the serum were analyzed using an automated serum analyzer (Dri-chem 2000, Fujifilm, Tokyo, Japan). As the enzymes in the serum, a total of six enzymes, including glutamate-pyruvate (GPT), glutamate-oxaloacetate (GOT), g-GT (gamma-phosphatase), alkaline phosphatase (AP), total bilirubin (TBIL) and direct-bilirubin (DBIL), were measured, and the results are shown in Tables below.

As enzymes used for diagnosis of liver disease, GOT, GPT, g-GT and ALP tests are frequently used. The increases in activity levels of these enzymes are not only highly correlated with the degree of cell disorder, but also are more sensitive to changes compared to those of other blood enzymes.

TABLE 4

| Classification | GOT (U/L) | GPT (U/L) |
|---|---|---|
| Control group | 75.4 ± 3.4 | 50.2 ± 3.6 |
| Group administered with | 121.2 ± 3.4 | 85.4 ± 3.1 |
| Comparative Example | | |
| Group administered with Example 1 | 88.4 ± 3.1 | 73.1 ± 2.7 |
| Group administered with Example 2 | 84.4 ± 4.4 | 70.2 ± 4.2 |
| Group administered with Example 3 | 78.2 ± 4.1 | 59.1 ± 4.4 |

Referring to Table 4 above, it could be confirmed that the activities of the enzymes significantly decreased in the groups administered with Examples 1 to 3 compared to the group administered with the Comparative Example.

In addition, the activity of alkaline phosphatase (AP) is shown in Table 5 below.

TABLE 5

| | AP |
|---|---|
| Classification | (U/L) |
| Control group | 150 |
| Group administered with | 275 |
| Comparative Example | |
| Group administered with Example 1 | 183 |
| Group administered with Example 2 | 181 |
| Group administered with Example 3 | 177 |
| Group administered with Example 4 | 179 |

Referring to Table 5 above, the groups administered with the Examples of the present invention showed an excellent effect corresponding to a decrease in AP activity of 31.2% on average in compared to the group administered with the Comparative Example. AP activity level rises as live cell damage/recovery cycles go up, and is an index that supports GPT inhibition. Effective inhibition of AP activity was found in the groups administered with alcohol+the granules prepared using the powder of the Example of the present invention. This suggests that the composition of the present invention has an activity of inhibiting the GOT or bilirubin activity.

Then, if gamma-glutamyl phosphatase (g-GT) is highly increased, it will indicate chronic cholestasis, and provide a putative diagnosis of biliary cirrhosis or sclerosing cholangitis. The results of analysis of g-GT activity are shown in Table 6 below.

TABLE 6

| | g-GT |
|---|---|
| Classification | (U/L) |
| Control group | 5.9 |
| Group administered with | 6.6 |
| Comparative Example | |
| Group administered with Example 1 | 5.5 |
| Group administered with Example 2 | 5.3 |
| Group administered with Example 3 | 4.9 |

Referring to Table 6 above, the groups administered with the Examples of the present invention showed an excellent effect corresponding to a decrease in g-GT activity of 20.6% on average compared to the group administered with the Comparative Example.

Lastly, when total bilirubin (TBIL) increases in the blood, it can indicate obstructive jaundice that is a condition in which there is a blockage of the flow of bile out of the liver. The results of analysis of TBIL activity are shown in Table 7 below.

TABLE 7

| | TBIL |
|---|---|
| Classification | (U/L) |
| Control group | 0.61 |
| Group administered with | 0.75 |
| Comparative Example | |
| Group administered with Example 1 | 0.52 |
| Group administered with Example 2 | 0.50 |
| Group administered with Example 3 | 0.42 |

Referring to Table 7 below, the groups administered with the Examples of the present invention showed an excellent effect corresponding to a decrease in TBIL of 39% on average compared to the group administered with the Comparative Example.

<Results of Histological Analysis>

The right lobe of the liver tissue of each experimental animal was taken, and the contents of reduced glutathione (GSH) and oxidized glutathione (GSSG), which are involved in the redox function, in the liver tissue, were measured.

The taken liver tissue was sectioned and weighed, and then a 4-fold amount of 0.1 M potassium phosphate buffer (pH 7.4) was added thereto. Then, each tissue sample was homogenized on ice with a tissue homogenizer to obtain an enzyme solution containing the enzymes. Using this enzyme solution, analysis of the content of reduced glutathione was performed according to the method of Ellman (1959) and S′wiergosz-Kowaleska et al. (2006). Specifically, total glutathione content was first analyzed, and then the content of reduced glutathione was determined by subtracting the content of oxidized glutathione from the total glutathione content. The results are shown in Table 8 below.

“Reduced glutathione” refers to glutathione having an SH (S: sulfur, and H: hydrogen) group attached thereto. This reduced glutathione serves to remove harmful reactive oxygen species. Referring to Table 8 below, the content of reduced glutathione increased by 14.5% on average in the groups administered with the Examples compared to the group administered with the Comparative Example. Thus, it could be confirmed that the compositions of the Examples are more effective for antioxidant activity.

TABLE 8

| | Total | Reduced |
|---|---|---|
| Classification | GSH (μmol) | GSH (μmol) |
| Control group | 4.66 ± 0.38 | 3.99 ± 0.35 |
| Group administered with | 4.28 ± 0.42 | 3.32 ± 0.33 |
| Comparative Example | | |
| Group administered with | 4.59 ± 0.24 | 37.5 ± 0.31 |
| Example 1 | | |
| Group administered with | 4.57 ± 0.41 | 38.0 ± 0.42 |
| Example 2 | | |
| Group administered with | 4.59 ± 0.42 | 38.5 ± 0.44 |
| Example 3 | | |

<Evaluation of Effects on Inhibition of Inflammation and Oxidative Stress>

In experiments, the inflammatory cytokine TNF-α and IL-1β were measured in blood from all animals, and the results are shown in Table 9 below.

In each experiment, analysis was performed using a kit (TNF-α IRTA800) provided from R&D systems. Briefly, the serum obtained in this experiment was incubated for 2 hours in a 96-well plate precoated with an antibody capable of binding to each cytokine present in blood, and was then incubated with secondary antibody. Then, each well was treated with a substrate solution capable of determining the amount of binding, and the absorbance at 540 nm or 570 nm was measured.

TABLE 9

| | TNF-α | IL-1β |
|---|---|---|
| Classification | (pg/ml) | (pg/ml) |
| Control group | 6.1 ± 0.2 | 131.2 ± 3.5 |
| Group administered with | 17.3 ± 1.7 | 196.3 ± 5.0 |
| Comparative Example | | |
| Group administered with | 8.5 ± 1.3 | 130.2 ± 2.8 |
| Example 1 | | |
| Group administered with | 8.3 ± 1.5 | 127.2 ± 5.2 |
| Example 2 | | |
| Group administered with | 7.7 ± 0.4 | 125.1 ± 3.1 |
| Example 3 | | |

Referring to Table 9 above, it could be seen that in the group administered with the Comparative Example, the expression of both TNF-α and IL-1β increased, but in the groups administered with the Examples, the expression of these cytokines and markers decreased significantly close to that in the control group, which is a normal diet group, and thus the production of inflammatory substances due to alcohol consumption was suppressed.

Through the above results, it can be seen that the composition of the present invention has a very excellent effect on liver protection. Due to this effect, the composition of the present invention may be commercially used as a pharmaceutical composition or a health functional food for preventing, ameliorating or treating various alcoholic diseases such as alcohol-induced hepatitis or fatty acid.

<Evaluation of Effect of Reducing Blood Alcohol Concentration>

To evaluate the alcohol degradation effect of the compositions prepared in the Examples, alcohol was administered to experimental animals, and after 1, 3 and 5 hours, the concentrations of alcohol and acetaldehyde in the blood were measured, thereby evaluating the in vivo alcohol degradation effects of the compositions. The number of the experimental animals was 6 animals per group, and the animals were divided into experiment groups as shown in Table 2 above.

For alcohol administration, a 25% (v/v) aqueous solution of ethanol was orally administered daily through the stomach tube at a level of 3 g/kg body weight/day. For administration of each sample, 500 mg/kg of the powder for granule preparation of each Example was dispersed in the same amount of distilled water as used for alcohol administration and was administered twice at 30 minutes before and after alcohol administration.

At 1 and 5 hours after administration, the concentrations of alcohol and acetaldehyde in the blood were measured, thereby evaluating the in vivo alcohol degradation effect of each composition. For measurement, blood was collected from the abdominal main vein of each experimental animal, and the concentration of alcohol in the serum obtained by centrifuging the collected blood for 30 minutes at 3000 rpm was measured using an ethanol measurement kit (Abcam AB6543). The results are shown in Table 10 below.

TABLE 10

| | | | Blood | |
|---|---|---|---|---|
| | Blood Alcohol | | acetaldehyde | |
| | concentration | | concentration | |
| | (mg % alcohol) | | (mg/L) | |
| | 1 | 5 | 1 | 5 |
| Group administered | 203.2 ± 4.1 | 148.2 ± 4.5 | 0.258 | 0.132 |
| with Comparative | | | | |
| Example | | | | |
| Group administered | 92.4 ± 5.1 | 77.2 ± 3.5 | 0.214 | 0.88 |
| with Example 1 | | | | |
| Group administered | 91.5 ± 3.5 | 74.8 ± 2.9 | 0.214 | 0.85 |
| with Example 2 | | | | |
| Group administered | 90.1 ± 4.1 | 64.8 ± 3.3 | 0.210 | 0.77 |
| with Example 3 | | | | |

Referring to Table 10 above, the blood alcohol concentration and acetaldehyde concentration in the group administered with the Comparative Example decreased over time following alcohol administration, but the degree of the decrease was moderate. However, it could be seen that in the group administered with alcohol+the composition of each Example, the blood alcohol concentration significantly decreased compared to that in the group administered with the Comparative Example, and the blood acetaldehyde concentration also significantly decreased. Thus, it was confirmed that the composition of the present invention has the effect of significantly reducing the blood ethanol and acetaldehyde concentrations by maximizing the ability to relive hangovers.

<Examination of Aldehyde Dehydrogenase (ALDH) Activity and Acetaldehyde (ADH) Production>

In order to examine the effect of the composition of each Example on aldehyde dehydrogenase (ALDH) activity and acetaldehyde (ADH) production, the activity of ALDH and the content of ADH were measured using the experimental animal serum obtained in Experimental Example 1. The ALDH activity was measured using an aldehyde dehydrogenase activity colorimetric assay kit (BioVision), and the ADH content was measured using an aldehyde quantification assay kit. The results are shown in Table 11 below.

TABLE 11

| | ALDH | | ADH | |
|---|---|---|---|---|
| | activity (mU) | | (mM/L) | |
| | 1 | 5 | 1 | 5 |
| Group administered with | 1.4 ± 0.8 | 1.1 ± 0.3 | 115 ± 8.2 | 147 ± 8.7 |
| Comparative Example | | | | |
| Group administered with | 1.8 ± 0.2 | 1.4 ± 0.3 | 105 ± 8.2 | 127 ± 8.4 |
| Example 1 | | | | |
| Group administered with | 1.8 ± 0.5 | 1.5 ± 0.1 | 101 ± 8.7 | 121 ± 8.4 |
| Example 2 | | | | |
| Group administered with | 1.9 ± 0.2 | 1.8 ± 0.4 | 94.5 ± 7.2 | 104 ± 8.1 |
| Example 3 | | | | |

Referring to Table 11 above, in the group administered with each Example, the aldehyde dehydrogenase activity was significantly higher and the acetaldehyde content also significantly decreased, compared to those in the group administered with the Comparative Example. Thus, it can be seen that the composition of the present invention has an excellent ability to degrade alcohol.

<Evaluation of Hangover Relieving Effect>

In order to confirm whether the composition of the present invention actually relieves a hangover the next day after drinking, the composition of the present invention was allowed to be taken after drinking.

An experiment was performed on each group consisting of 10 healthy men and women over 20 years of age. For the experiment, the test subjects were allowed to drink 300 ml of soju over 60 minutes while they were allowed to take three pieces of pork belly per cup of soju. Immediately after drinking, they were allowed to take 300 ml of each of the Example and Comparative Example compositions shown in Table 1 above. Group 2 administered with the Comparative Example was allowed to take two bottles of commercially available MorningCare® (200 ml). As a control, 300 ml of mineral water was used.

The hangover relieving effect, digestive effect and headache level presented by these groups the next morning were evaluated on a 5-point scale: 1=not effective; and 5=most highly effective. The results are shown in Table 12 below.

TABLE 12

| | | | Degree |
|---|---|---|---|
| | Hangover | Degree | of |
| | relieving | of | headache |
| | effect | digestion | reduction |
| Control group | 1.1 | 1.2 | 1.1 |
| Group 1 administered with | 3.1 | 3.1 | 3.0 |
| Comparative Example | | | |
| Group 2 administered with | 2.8 | 2.9 | 3.1 |
| Comparative Example | | | |
| Example 1 | 3.7 | 3.6 | 3.7 |
| Example 2 | 3.9 | 3.8 | 4.1 |
| Example 3 | 4.4 | 4.3 | 4.5 |
| Example 4 | 4.1 | 4.1 | 4.1 |

Referring to Table 12 above, it could be seen that the group administered with the composition according to the present invention showed a very high effect on hangover relief, and in particular, this hangover relief effect was superior even to that in group 1 administered with the Comparative Example.

As described above, the composition of the present invention as described above has a liver damage suppressing effect, reduces hepatitis activity index, and thus may be used as a composition capable of preventing or treating various liver diseases. In addition, it has very excellent effects on alcohol degradation and acetaldehyde degradation, alleviates headache and vomiting symptoms the next day after drinking, and thus is also very useful as a composition for liver protection or hangover relief, which is taken before and after alcohol drinking.

1. A drink composition for preventing alcoholic liver disease or relieving hangover, which contains 2 to 4 wt % of black ginseng, 4 to 7 wt % of blueberry, 0.3 to 1 wt % of dry yeast, 0.5 to 1.5 wt % of jujube pulp, and 2 to 4 wt % of oriental raisin tree fruit and is produced by hot-water extraction using a remaining wt % of purified water.

2. The drink composition of claim 1, which further contains 2 to 4 wt % of green onion root and is produced by hot-water extraction.

3. The drink composition of claim 1, which further contains 2 to 4 wt % of radish greens and is produced by hot-water extraction.

4. The drink composition of claim 1, wherein the alcoholic liver disease is alcoholic hepatitis, alcoholic fatty liver, or alcoholic liver cirrhosis.

5. The drink composition of claim 2, which further contains 2 to 4 wt % of radish greens and is produced by hot-water extraction.

6. The drink composition of claim 2, wherein the alcoholic liver disease is alcoholic hepatitis, alcoholic fatty liver, or alcoholic liver cirrhosis.
